(19) United States
(12) Patent Application Publication
(10) Pub. No.: US 2022/0098457 A1
(43) Pub. Date: Mar. 31, 2022

(54) ELASTOMERIC ADHESIVES COMPRISING BIO-DERIVED COMPOUNDS

(71) Applicant: Zymergen Inc., Emeryville, CA (US)

(72) Inventors: Nandita Bhagwat, Emeryville, CA (US); John Dickens, Emeryville, CA (US); Joachim C. Ritter, San Rafael, CA (US); Gerald A. Vandezande, Raleigh, NC (US); Jonathon E. Vandezande, Raleigh, NC (US); John J. McNamara, El Sobrante, CA (US)

(21) Appl. No.: 17/424,082

(22) PCT Filed: Jan. 21, 2020
(86) PCT No.: PCT/US2020/014467

§ 371 Date: Jul. 19, 2021

Related U.S. Application Data
(60) Provisional application No. 62/795,473, filed on Jan. 22, 2019.

(51) Int. Cl. C09J 175/04; C08G 18/38

(52) U.S. Cl. CPC C09J 175/04; C08G 18/3821

(57) ABSTRACT

The present disclosure addresses elastomeric adhesives and elastomers that include a modified amine. The modified amine can be the reaction product of an amine with a Michael acceptor. The amine can be represented by H₂N—Ar—R¹—NH₂, wherein Ar is an arylene and R1 is selected from an alkylene and alkenylene. The Michael acceptor can be selected from a variety of compounds including maleates, fumarates, acrylates, and others.

### CROSS-REFERENCE TO RELATED APPLICATIONS

This application claims the benefit of U.S. provisional application No. 62/795,473, filed Jan. 22, 2019, which is hereby incorporated by reference in its entirety.

### FIELD OF THE DISCLOSURE

The present disclosure relates generally to the area of elastomers and elastomeric adhesives. More specifically, this disclosure relates to novel elastomeric resins formed by reaction products from bio-derived amines and Michael acceptors and polyisocyanates. Said resin can find application as adhesives, sealants, coatings, and other applications.

### BACKGROUND

There is a continuous need for new oligomeric and polymeric materials, which exhibit advantages offered by commercially available materials, while featuring other beneficial properties such as environmental compatibility, low toxicity, and minimal health impact. In addition, there is a need for new (co)polymers derivable from bio-based resources.

The use of conventional polyaspartates in coating compositions leads to certain disadvantages, including inadequate chemical resistance and the need for solvent to give an adequate pot-life. The choice of polyaspartate can affect cure rates, and can be difficult because certain applications require the use of solvent, which is to be avoided. Additionally, coatings generally suffer from various disadvantages, such as embrittlement of thick section coating, which leads to shrinkage and cracking, thereby creating leaks for moisture and air. Accordingly, there is a need to overcome such disadvantages.

### SUMMARY OF THE INVENTION

In a first aspect, an elastomeric adhesive includes a modified amine. The modified amine can be the reaction product of an amine with a Michael acceptor. The amine can be represented by

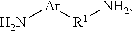

wherein
Ar is an arylene and R¹ is selected from an alkylene and alkenylene.

The Michael acceptor is selected from:

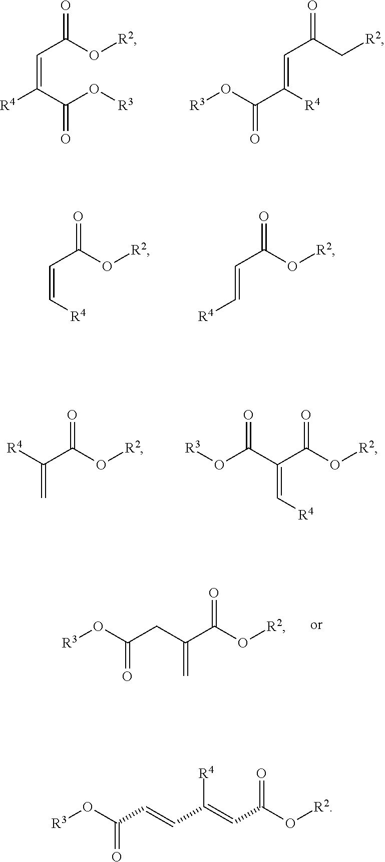

R² and R³ can for each occasion independently be selected from hydrogen, an alkyl or an alkenyl, R⁴ can for each occasion independently be selected from hydrogen, an alkyl, an alkenyl, an alkoxy, or an alkenoxy. In addition, the elastomeric adhesive includes an isocyanate resin.

In a second aspect, a method for adhering a first substrate and a second substrate includes applying the elastomeric adhesive to the first substrate. The method can further include contacting a second substrate to the elastomeric adhesive. The first substrate and the second substrate are independently selected from metal, glass, ceramic, or plastic.

In a third aspect, a two-component adhesive comprises a component A and a component B, Component A comprises:

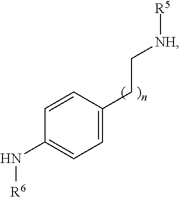

wherein R⁵ is selected from

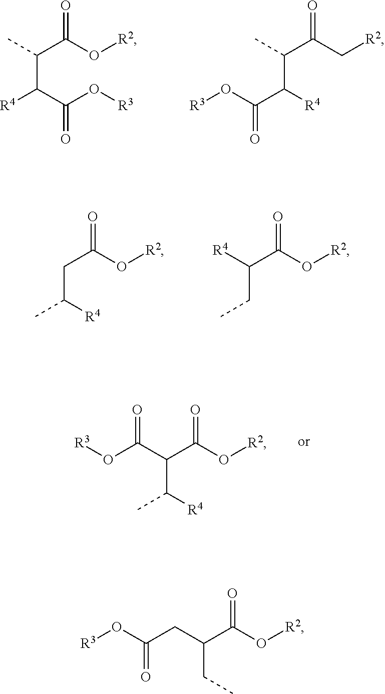

wherein R⁶ is selected from hydrogen,

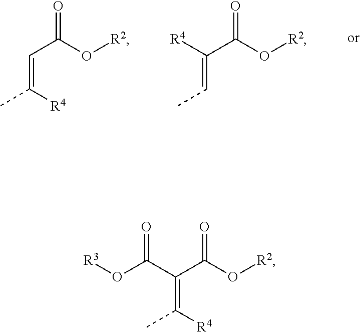

wherein n can be 0, 1, or 2, wherein R² and R³ are for each occasion independently selected from hydrogen, an alkyl, or an alkenyl, R⁴ is for each occasion independently selected from hydrogen, an alkyl, an alkenyl, an alkoxy, or an alkenoxy.

### DETAILED DESCRIPTION

This written description uses examples to disclose the embodiments, including the best mode, and also to enable those of ordinary skill in the art to make and use the invention. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

Note that not all of the activities described above in the general description or the examples are required, that a portion of a specific activity may not be required, and that one or more further activities may be performed in addition to those described. The order in which activities are listed is not necessarily the order in which they are performed.

In this specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive—or and not to an exclusive—or. For example, a condition A or B is satisfied by any one of the following: A is true (or present) and B is false (or not present), A is false (or not present) and B is true (or present), and both A and B are true (or present).

Benefits, other advantages, and solutions to problems have been described herein with regard to specific embodiments. However, the benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range.

As a stated in the Summary of the Invention, an elastomeric adhesive includes a modified amine. The modified amine can be the reaction product of an amine with a Michael acceptor. The amine can be represented by

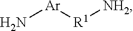

wherein
Ar is an arylene and R¹ is selected from an alkylene and alkenylene.

The Michael acceptor is selected from:

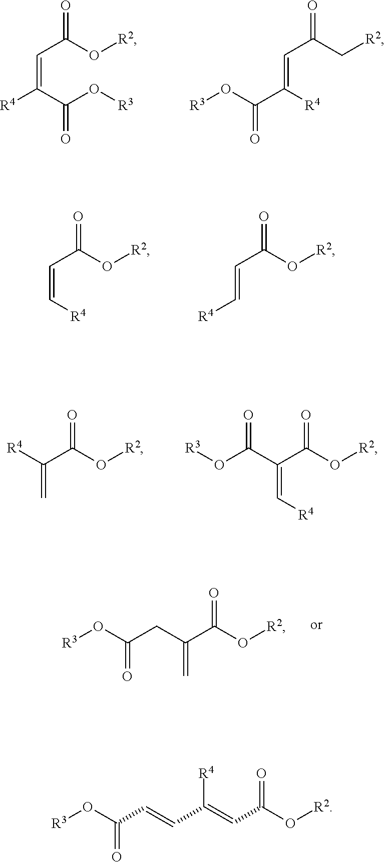

R² and R³ can for each occasion independently be selected from hydrogen, an alkyl, or an alkenyl, R⁴ can for each occasion independently be selected from hydrogen, an alkyl, an alkenyl, an alkoxy, or an alkenoxy. In addition, the elastomeric adhesive can further include an isocyanate resin.

In one embodiment, the elastomeric adhesive can further include modified amines, wherein Ar can be selected from phenylene, benzene, naphthalene, biphenyl, phenoxyphenyl, 4′-ethylenoxy-phenyl-4-ethyl anthracene, terphenyl, fluorene, pyridine, 1,2-diazine, 1,3-diazine, 1,4-diazine, 1,2,3-triazine, 1,2,4-trazine, 1,3,5-triazine, purine, pyrrole, furan, thiophene, imidazole, pyrazole, 1H-1,2,3-triazole, 2H-1,2,3-triazole, 1H-1,2,4-triazole, or 4H-1,2,4-triazole.

In another embodiment, R¹ can be selected from methylene, ethylene, n-propylene, oxy-methylene, oxy-ethylene, -oxy-n-propylene, iso-propylene, n-butylene, cis-butenylene, trans-butenylene, butadienylene, polybutadienylene, iso-butylene, sec-butylene, tert-butylene, n-pentylene, iso-pentylene, neo-pentylene, cis-pentylene, trans-pentylene, cis,cis-1,3-penadienylene, cis, trans-1,3-pentadienylene, trans, trans-1,3-pentadienylene, isoprenylene, polyisoprenylene, n-hexanylene, iso-hexanylene, 3-methylpentanylene, neo-hexanylene, 2,3-dimethylbutanylene, 2-methylhexanylene, 2-ethylhexanylene, 2-propylhexanylene, hexenylene, hexadienylene, or hexatrieneylene.

In a further embodiment, R² and R³ can be independently selected from methyl, ethyl, n-propyl, iso-propyl, n-butyl, cis-butenyl, trans-butenyl, butadienyl, polybutadienyl, iso-butyl, sec-butyl, tert-butyl, n-pentyl, iso-pentyl, neo-pentyl, cis-pentyl, trans-pentyl, cis,cis-1,3-penadienyl, cis, trans-1,3-pentadienyl, trans, trans-1,3-pentadienyl, isoprenyl, polyisoprenyl, n-hexanyl, iso-hexanyl, 3-methylpentanyl, neo-hexanyl, 2,3-dimethylbutanyl, 2-methylhexanyl, 2-ethylhexanyl, 2-propylhexanyl, hexenyl, hexadienyl, or hexatrieneyl.

A Michael acceptor is a compound comprising a carbon-carbon unsaturation between the alpha and beta carbons adjacent to an electron withdrawing group such as a keto or carboxy group. For example, Michael acceptors are esters of the following acids: acrylic acid, methacrylic acid, crotonic acid, maleic acid, fumaric acid, mesaconic acid, citraconic acid, itaconic acid, and muconic acid.

In one embodiment, the amine or the Michael acceptor are bio-based and can be obtained from biological preparation, e.g., fermentation. In one further embodiment, the foregoing amine, Michael acceptor, or adhesive has a bio-based carbon content of at least 2%, such as at least 5%, at least 10%, at least 15%, at least 20%, or at least 25% as determined by ASTM D6866. Bio-based carbon content as defined herein is the percentage of carbons from renewable or biogenic sources, such as plants or animals over the total number of carbons in the compound.

In one embodiment, the amine of the adhesive as represented by

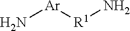

can be selected from:

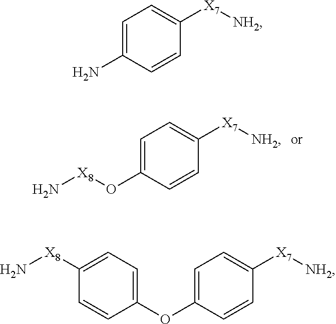

wherein X₇ and X₈ is for each occasion independently selected from methylene, ethylene, n-propylene, iso-propylene, n-butylene, or sec-butylene.

In one embodiment, Ar in the foregoing structure can be selected from phenylene, benzene, naphthalene, biphenyl, phenoxyphenyl, 4′-ethylenoxy-phenyl-4-ethyl anthracene, terphenyl, fluorene, pyridine, 1,2-diazine, 1,3-diazine, 1,4-diazine, 1,2,3-triazine, 1,2,4-trazine, 1,3,5-triazine, purine, pyrrole, furan, thiophene, imidazole, pyrazole, 1H-1,2,3-triazole, 2H-1,2,3-triazole, 1H-1,2,4-triazole, or 4H-1,2,4-triazole.

In one embodiment, the elastomeric adhesive can further include a second reaction product of an aliphatic amine and the Michael acceptor. For such an embodiment, the aliphatic amine can be selected from putrescine, cadaverine, norspermindine, spermine, norspermine, spermidine, diethylenetriamine, triethylenetetramine, tris(2-aminoethyl)amine, cyclen, 1,4,7-Triazacyclononane, or 1,1,1-Tris(aminomethyl)ethane.

In yet another embodiment, the elastomeric adhesive can include that the amine or the aliphatic amine is produced by way of fermentation from gram positive bacteria, gram negative bacteria, fungi, and yeast.

In one further embodiment, the isocyante resin of the elastomeric adhesive can include hexamethylene diisocyanate, isophorone diisocyanate, 4,4-diisocyanatodicyclohexylmethane, 1,4-diisocyanatocyclohexane, 1-methyl-2,4-diisocyanatocyclohexane, 1-methyl-2,6-diisocyanatocyclohexane, 4-isocyanatomethyl-1,8-octane diisocyanate, toluene diisocyanate, methylene diphenyl diisocyanate, a isocyanurate trimers, a biurete, a uretdione dimers, or any combination thereof.

The isocyanate resins of the aforementioned kind preferably have an NCO group content of 5 to 25% by weight, an average NCO functionality of 2.0 to 5.0, preferably 2.8 to 4.0, and a residual amount of monomeric diisocyanates, used for their preparation, of below 1% by weight, preferably below 0.5% by weight.

In one embodiment, the elastomeric adhesive comprises at least one property, such as at least two, or at least three properties selected from:
a. an elongation, as defined in ASTM D882-18, of a cured adhesive is between 10% and 700%, between 10% and 500%, between 10% and 300%, between 10% and 200%, between 10% and 100%, and between 10% and 50%;
b. a crosshatch adhesion, as defined in ASTM D3359-17, to metal, glass, ceramic, and plastic is greater than 4B;
c. a pencil hardness, as defined in ASTM D3363-05, of the cured adhesive is between 3B and 7H, between 2B and 6H, between 1B and 5H;
d. a yellowness index, as defined in ASTM E313-15, of the cured adhesive is less than 2; less than 1.9, less than 1.8, less than 1.7, less than 1.6, less than 1.5, less than 1.4, less than, 1.3, less than 1.2, less than 1.1, less than 1.0, less than 0.9, or less than 0.8;
e. a tensile modulus, as defined in ASTM D638-14, of the cured adhesive is greater than 0.1 MPa, greater than 0.2 MPa, greater than 0.5 MPa, greater than 1 MPa, greater than 2 MPa, greater than 5 MPa, greater than 10 MPa, greater than 20 MPa, greater than 50 MPa, greater than 60 MPa, greater than 80 MPa, greater than 100 MPa, greater than 110 MPa, greater than 120 MPa, greater than 130 MPa, greater than 140 MPa, greater than 150 MPa, greater than 200 MPa, or greater than 250 MPa;
f. a solvent resistance, as defined in ASTM D5402-15, Method B, of the cured adhesive greater than 10 rubs, greater than 20 rubs, greater than 30 rubs, greater than 40 rubs, greater than 50 rubs, greater than 60 rubs, greater than 80 rubs, greater than 100 rubs, greater than 120 rubs, greater than 140 rubs, greater than 160 rubs, greater than 180 rubs, or greater than 200 rubs.
g. a glass transition temperature as determined by Differential Scanning calorimetry of greater than −40° C., greater than −30° C., greater than −20° C., greater than −10° C., greater than 0° C., greater than 10° C., greater than 20° C., or greater than greater than 30° C.;
h. a lap shear strength as determined by ASTM D1002 of greater than 50 psi, greater than 80 psi, greater than 100 psi, greater than 200 psi, greater than 500 psi, or greater than 750 psi; and/or
i. a pot life of greater than 30 seconds, greater than 1 minute, greater than 2 minutes, greater than 4 minutes, greater than 6 minutes, greater than 8 minutes, or greater than 10 minutes.

Pot life is defined as the amount of time it takes for an initial mixed viscosity to reach 1000 cP. Timing starts from the moment the product is mixed, and is measured at room temperature (23° C.)

In one embodiment, the elongation at break is not greater than 700%, the crosshatch adhesion is 5B, the pencil hardness is not greater than 9H and not softer than 9B, the yellow index is at least 0.05, the tensile modulus is not greater than 1 GPa, or the solvent resistance is not greater than 1000 rubs.

In one embodiment, the elastomeric can further include a solvent. The solvent can be selected from a polar protic solvent or a polar aprotic solvent. Suitable solvents can be methanol, ethanol, propanol, diethylether, tetrahydrofuran, dioxane, cyclohexanone, ethyl acetate, or mixtures thereof.

In one embodiment, the elastomeric adhesive can further include an additive. Such additives include surface modifier, e.g., silicone surface modifiers, fillers, e.g., silica or carbon based fillers, such as carbon nanotubes, metal filings, powders, pigments, pigment stabilizers, defoamers, adhesion promoters, e.g., silane based adhesion promoters, acid based adhesion promoters or alcohol based adhesion promoters, chain extenders, e.g., polyethylene glycols, thickeners, e.g., cellulose or clays, UV-absorbants.

In one embodiment, a method for adhering a first substrate and a second substrate can include applying the elastomeric adhesive onto the first substrate. If the adhesive is cured, it serves as a coating to the substrate. In another embodiment, the method can further include contacting a second substrate to the elastomeric adhesive. The first substrate and the second substrate can be independently selected from metal, glass, ceramic, or plastic.

As described in the Summary of the Invention, a two-component adhesive comprises a component A and a component B, Component A comprises:

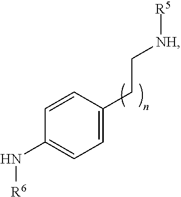

wherein R⁵ is selected from

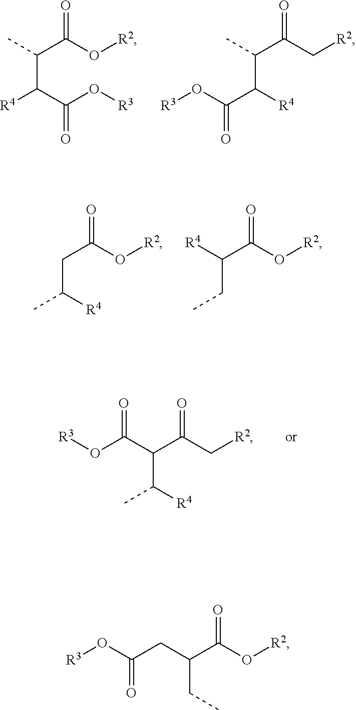

wherein R⁶ is selected from hydrogen,

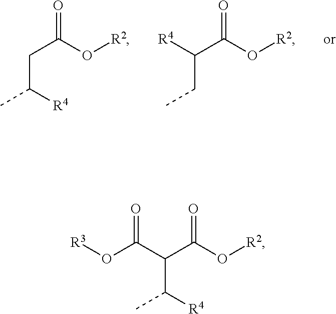

wherein n can be 0, 1, and 2, wherein R² and R³ are for each occasion independently selected from hydrogen, an alkyl, or an alkenyl, R⁴ is for each occasion independently selected from hydrogen, an alkyl, an alkenyl, an alkoxy, or an alkenoxy.

In one embodiment, the amine can be derived from p-aminophenylethylamine (APEA), p-aminophenylmethylamine (APMA), or p-aminophenylpropylamine (APPA).

In another embodiment, the substrate can be selected from metal, glass, ceramic, or plastic. In yet another embodiment, the metal can be selected from cold rolled steel, stainless steel, aluminum, anodized aluminum, nickel, alloys, electroless plated metal, or electroplated metal. In one further embodiment, the glass can be selected from borosilicate glass or quartz. In one more embodiment, the plastic can be selected from polycarbonate, polyethylene terephthalate, polyamide, filled polyamide, polyester, poly(methyl methacrylate), polyacrylates, polystyrene, polyvinyl, chlorinated polyvinyl, or laminates thereof.

In one further embodiment, the elastomer of the article can have a water vapor permeability according to ASTM D1653-13 of not more than 100 g (m² 24 h)⁻¹, not more than 80 g (m² 24 h)⁻¹, not more than 60 g (m² 24 h)⁻¹, not more than 40 g (m² 24 h)⁻¹, not more than 20 g (m² 24 h)⁻¹, not more than 15 g (m² 24 h)⁻¹, not more than 10 g (m² 24 h)⁻¹, not more than 8 g (m² 24 h)⁻¹, not more than 5 g (m² 24 h)⁻¹, not more than 3 g (m² 24 h)⁻¹, not more than 2 g (m² 24 h)⁻¹, not more than 1 g (m² 24 h)⁻¹, not more than 0.8 g (m² 24 h)⁻¹, not more than 0.5 g (m² 24 h)⁻¹, not more than 0.2 g (m² 24 h)⁻¹, not more than 0.1 g (m² 24 h)⁻¹, or not more than 0.05 g (m² 24 h)⁻¹. In one embodiment, the water permeability is at least 0.0001 mg (m² 24 h)⁻¹.

In yet another embodiment, the article comprising the elastomer as a coating can be an electronic wearable, a handheld electronic consumer devices, an electronic appliance, a marine electronic device, an acoustic device, a snow vehicle, a water vehicle, or a liquid container.

In one embodiment the article includes an elastomer that has an air permeability according to ASTM D2357-18 of not more than 50 mL (m² s)⁻¹, not more than 30 mL (m² s)⁻¹, not more than 20 mL (m² s)⁻¹, not more than 10 mL (m² s)⁻¹, not more than 5 mL (m² s)⁻¹, not more than 2 mL (m² s)⁻¹, not more than 1 mL (m² s)⁻¹, not more than 0.8 mL (m² s)⁻¹, not more than 0.5 mL (m² s)⁻¹, not more than 0.2 mL (m² s)⁻¹, not more than 0.1 mL (m² s)⁻¹. In one embodiment, the air permeability is at least 0.0001 microL (m² s)⁻¹.

In yet one further embodiment, the elastomer has a transmittance according to ASTM standard D1746-15 of at least 60%, at least 65%, at least 70%, at least 75%, at least 80%, at least 85%, at least 90%, at least 92%, at least 93%, or at least 94%. In one embodiment, the transmittance is not greater than 99.9%

In further embodiments, the elastomeric adhesive as described herein is a composition that is a 100% resin composition which does not contain any solvents. Accordingly, the adhesive can be a zero VOC adhesive.

In yet one further embodiment, for use of the adhesive as a coating or adhesives, it can be applied on flexible substrates. Flexible substrates include silicon elastomers, rubber, thermoplastic polyurethanes, and thermoplastic elastomers. In another embodiment, when the adhesive is used as a coating, it can be a haptic coating, i.e., a coating that provides a soft or velvet feel. The adhesive can be applied using spray coating, doctor blading, or roll coating processes.

In one embodiment, the amine and a maleate ester are reacted first to form a modified amine as shown here:

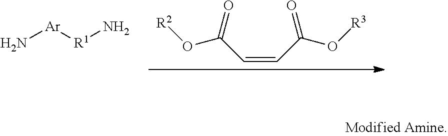

More specific example is the following reaction:

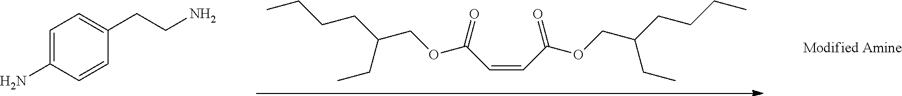 [SMILES: NCCc1ccc(N)cc1 ; CCCCC(CC)COC(=O)/C=C\C(=O)OCC(CC)CCCC]

The modified amine includes the Michael adduct, there by forming an aspartate that can further polymerize to a polyaspartate. Next, the isocyanate resin or polyisocyanate is added which reacts with free amines to form a polyuria and the composition in its entirety is the elastomeric adhesive, as shown here:

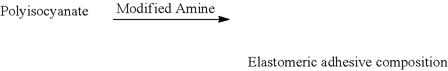

Experimental:

Synthesis of modified amines:

Diethylhexylmaleate (DEHM) was mixed with p-aminophenylethylamine (APEA) in a ratio of 1:1. The mixture was monitored by GCMS, HPLC, and NMR to obtain more than 90% yield of the Michael adduct. The crude product can be purified by standard techniques such as flash chromatography over silicagel or recrystallization in ethyl acetate to form the DEHM:APEA adduct:

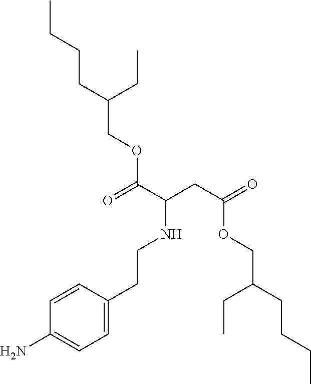 [SMILES: CCCCC(CC)COC(=O)CC(NCCc1ccc(N)cc1)C(=O)OCC(CC)CCCC]

In another experiment, APEA and DEHM were reacted together, and after 24 hours, 1 equivalent of butyl acrylate (BA) was added and either a stoichiometric amount of acetic acid or a catalytic amount (1 to 5 mol %) of a Lewis acid such as SnCl₂ or FeCl₃. The mixture was heated to 100° C. for another 24 hours and monitored by GCMS, HPLC, and NMR. The crude product can be purified by standard techniques such as flash chromatography over silicagel or recrystallization in ethyl acetate. After isolation, the DEHM:APEA:BA adduct was formed:

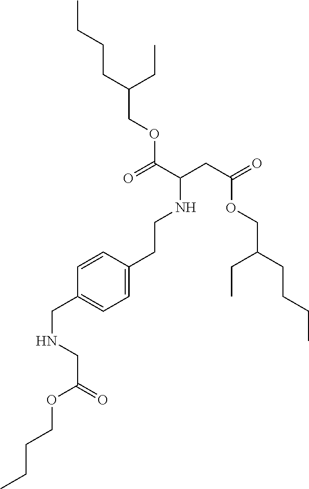 [SMILES: CCCCOC(=O)CNCc1ccc(CCNC(CC(=O)OCC(CC)CCCC)C(=O)OCC(CC)CCCC)cc1]

The same procedure was used for the reaction of dimethyl citraconic acid (DMC) and APEA to form DMC:APEA. The reaction was monitored by GCMS, HPLC, and NMR, and the temperature was raised to 40° C. over the course of 24 to 48 hours.

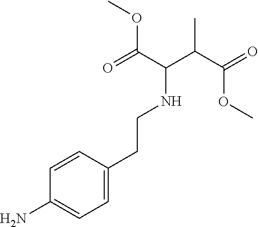 [SMILES: COC(=O)C(C)C(NCCc1ccc(N)cc1)C(=O)OC]

The foregoing procedure was used for the reaction of dimethyl methoxy-methylene-malonate (DM-MMM) and APEA to form a mixture of the Michael adduct and its demethoxylated product:

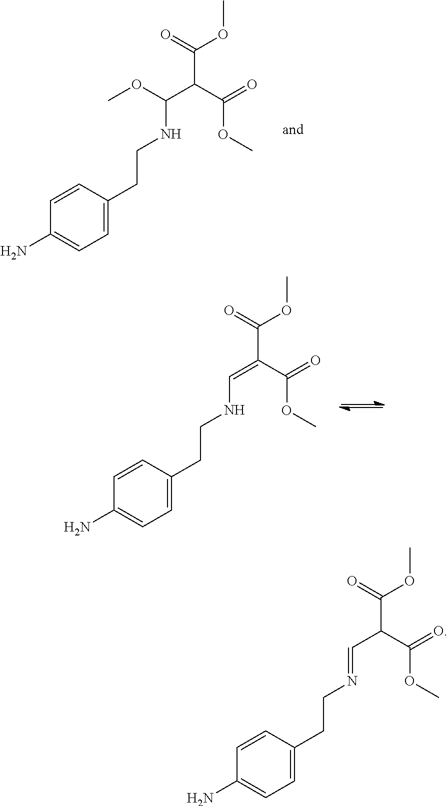 [SMILES: COC(=O)C(C(=O)OC)C(NCCc1ccc(N)cc1)OC ; COC(=O)C(=CNCCc1ccc(N)cc1)C(=O)OC ; COC(=O)C(/C=N/CCc1ccc(N)cc1)C(=O)OC]

Elastomeric Adhesive Formulation

The Michael adducts were mixed neat, or with up to 10 wt % of cyclohexanone or ethyl acetate, with a isocyanate and applied to a substrate, such as metal (stainless steel) or plastic (Lexan or Kalix). The amount of each component to achieve an amino:isocyanate ratio of approx. 1:1. The applied mixture was cured in an oven at between 70° C. and 200° C. dependent on the nature and heat stability of the substrate. The degree of curing was sporadically checked by testing tackiness of the applied mixture or by monitoring the reduction of isocyanate via FTIR. Isocyanates used were:

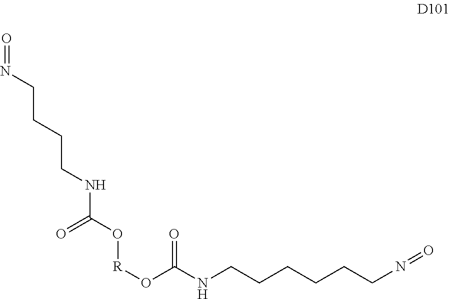

wherein R is alkylene, such as 1,2-ethylene or 1,2,3-propylene.

Table 1 displays the formulations prepared and Table 2 the properties obtained.

TABLE 1

| Form. | Aspartate | Isocyanate | Solvent |
|---|---|---|---|
| A | DEHM-APEA | E402-100 | Cyclohexanone |
| B | DEHM-APEA | N3400, E402-100 (1:1) | Cyclohexanone |
| C | DEHM-APEA | D101 | Cyclohexanone |
| D | DEHM-APEA-BA | N3400, E402-100 (1:1) | Cyclohexanone |

TABLE 2

| | | | Cross | Pencil | Elongation | T_{g}/ | Lap |
|---|---|---|---|---|---|---|---|
| Form. | Pot Life | | Hatch | Hardness | at break | ° C. | Shear/psi |
| A | 1 | min | 5b | 3H | 250% | −25 | 50-100 |
| B | 1 | min | 5b | 3H | 230% | 5 | 200-500 |
| C | 1 | min | 5b | 4H | 270% | 34 | 350-500 |
| D | 5-10 | min | 5b | 3H | 120% | | |

As can be seen from Table 2, the bio-based elastomeric adhesives show that valuable properties such as low T_{g}, high elongation, and improved hardness can be obtained with variable pot life.

1. An elastomeric adhesive comprising of
a. a modified amine comprising of the reaction product of an amine with a Michael acceptor, wherein the amine is represented by 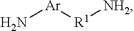
wherein Ar is an arylene, R¹ is selected from an alkylene and alkenylene, and the Michael acceptor is selected from the group consisting of: 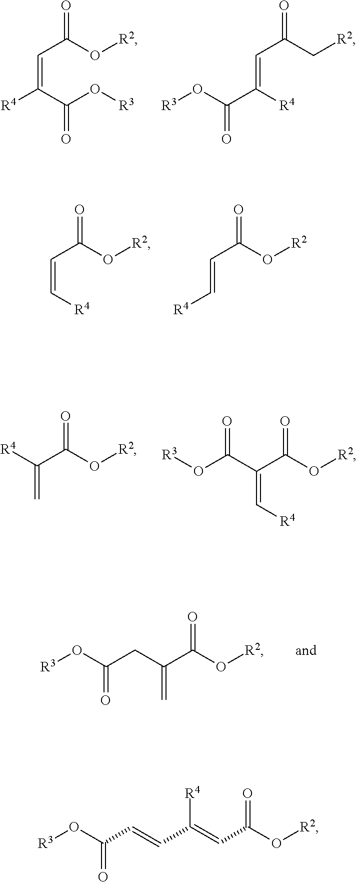
wherein
R² and R³ are for each occasion independently selected from hydrogen, an alkyl, or an alkenyl, R⁴ is for each occasion independently selected from hydrogen, an alkyl, an alkenyl, an alkoxy, or an alkenoxy; and
b. an isocyanate resin.

2. The elastomeric adhesive according to claim 1 comprising at least one property, at least two properties, or at least three properties selected from
a. an elongation at break, as defined in ASTM D882-18, of a cured adhesive is between 10% and 700%, between 10% and 500%, between 10% and 300%,between 10% and 200%, between 10% and 100%, or between 10% and 50%;
b. a crosshatch adhesion, as defined in ASTM D3359-17, to metal, glass, ceramic, and plastic is greater than 4B;
c. a pencil hardness, as defined in ASTM D3363-05, of the cured adhesive is between 3B and 7H, between 2B and 6H, between 1B and 5H;
d. a yellowness index, as defined in ASTM E313-15, of the cured adhesive is less than 2; less than 1.9, less than 1.8, less than 1.7, less than 1.6, less than 1.5, less than 1.4, less than, 1.3, less than 1.2, less than 1.1, less than 1.0, less than 0.9, or less than 0.8;
e. a tensile modulus, as defined in ASTM D638-14, of the cured adhesive is greater than 0.1 MPa, greater than 0.2 MPa, greater than 0.5 MPa, greater than 1 MPa, greater than 2 MPa, greater than 5 MPa, greater than 10 MPa, greater than 20 MPa, greater than 50 MPa, greater than 60 MPa, greater than 80 MPa, greater than 100 MPa, greater than 110 MPa, greater than 120 MPa, greater than 130 MPa, greater than 140 MPa, greater than 150 MPa, greater than 200 MPa, or greater than 250 MPa;
f. a solvent resistance, as defined in ASTM D5402-15, Method B, of the cured adhesive greater than 10 rubs, greater than 20 rubs, greater than 30 rubs, greater than 40 rubs, greater than 50 rubs, greater than 60 rubs, greater than 80 rubs, greater than 100 rubs, greater than 120 rubs, greater than 140 rubs, greater than 160 rubs, greater than 180 rubs, or greater than 200 rubs;
g. a glass transition temperature as determined by Differential Scanning calorimetry of greater than −40° C., greater than −30° C., greater than −20° C., greater than −10° C., greater than 0° C., greater than 10° C., greater than 20° C., or greater than greater than 30° C.;
h. a lap shear strength as determined by ASTM D1002 of greater than 50 psi, greater than 80 psi, greater than 100 psi, greater than 200 psi, greater than 500 psi, or greater than 750 psi; or
i. a pot life of greater than 30 seconds, greater than 1 minute, greater than 2 minutes, greater than 4 minutes, greater than 6 minutes, greater than 8 minutes, or greater than 10 minutes.

3. The elastomeric adhesive according to claim 1, wherein
Ar is selected from phenylene, benzene, naphthalene, biphenyl, phenoxyphenyl, 4′-ethylenoxy-phenyl-4-ethyl anthracene, terphenyl, fluorene, pyridine, 1,2-diazine, 1,3-diazine, 1,4-diazine, 1,2,3-triazine, 1,2,4-trazine, 1,3,5-triazine, purine, pyrrole, furan, thiophene, imidazole, pyrazole, 1H-1,2,3-triazole, 2H-1,2,3-triazole, 1H-1,2,4-triazole, or 4H-1,2,4-triazole;
R¹ is selected from methylene, ethylene, n-propylene, oxy-methylene, oxy-ethylene, -oxy-n-propylene, iso-propylene, n-butylene, cis-butenylene, trans-butenylene, butadienylene, polybutadienylene, iso-butylene, sec-butylene, tert-butylene, n-pentylene, iso-pentylene, neo-pentylene, cis-pentylene, trans-pentylene, cis,cis-1,3-penadienylene, cis, trans-1,3-pentadienylene, trans, trans-1,3-pentadienylene, isoprenylene, polyisoprenylene, n-hexanylene, iso-hexanylene, 3-methylpentanylene, neo-hexanylene, 2,3-dimethylbutanylene, 2-methylhexanylene, 2-ethylhexanylene, 2-propylhexanylene, hexenylene, hexadienylene, or hexatrieneylene; and
R² and R³ is independently selected from methyl, ethyl, n-propyl, iso-propyl, n-butyl, cis-butenyl, trans-butenyl, butadienyl, polybutadienyl, iso-butyl, sec-butyl, tert-butyl, n-pentyl, iso-pentyl, neo-pentyl, cis-pentyl, trans-pentyl, cis,cis-1,3-penadienyl, cis, trans-1,3-pentadienyl, trans, trans-1,3-pentadienyl, isoprenyl, polyisoprenyl, n-hexanyl, iso-hexanyl, 3-methylpentanyl, neo-hexanyl, 2,3-dimethylbutanyl, 2-methylhexanyl, 2-ethylhexanyl, 2-propylhexanyl, hexenyl, hexadienyl, or hexatrieneyl.

4. The elastomeric adhesive according to claim 1, further comprising a second reaction product of an aliphatic amine and the Michael acceptor.

5. The elastomeric adhesive according to claim 4 wherein the aliphatic amine is selected from putrescine, cadaverine, norspermindine, spermine, norspermine, spermidine, diethylenetriamine, triethylenetetramine, tris(2-aminoethyl)amine, cyclen, 1,4,7-Triazacyclononane, or 1,1,1-Tris(aminomethyl)ethane.

6. The elastomeric adhesive according to claim 1, wherein the amine or the aliphatic amine is produced by way of fermentation from gram positive bacteria, gram negative bacteria, fungi, and yeast.

7. The elastomeric adhesive according to claim 1, wherein the isocyante resin comprises hexamethylene diisocyanate, isophorone diisocyanate, toluene diisocyanate, methylene diphenyl diisocyanate, an isocyanurate trimers, a biurete, a uretdione dimers, or any combination thereof.

8. The elastomeric adhesive according to claim 1, wherein 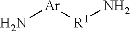
is selected from 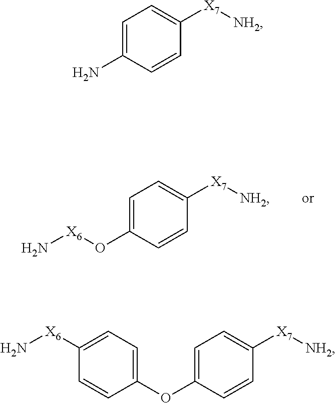
wherein
X₇ and X₈ is for each occasion independently selected from methylene, ethylene, n-propylene, iso-propylene, n-butylene, or sec-butylene.

9. The elastomeric adhesive according to claim 1, further comprising a solvent, wherein the solvent is selected from a polar protic solvent or a polar aprotic solvent.

10. Use of an elastomeric adhesive according to claim 1 for coating flexible substrates.

11. The use according to claim 10, wherein the flexible substrate is selected from silicon elastomers, rubber, thermoplastic polyurethanes, or thermoplastic elastomers.

12. A method for adhering a first substrate and a second substrate comprising
applying the elastomeric adhesive according to claim 1 to the first substrate, and
contacting a second substrate to the elastomeric adhesive; wherein the first substrate and the second substrate are independently selected from metal, glass, ceramic, or plastic.

13. A two-component adhesive comprising a component A and a component B, wherein component A comprises: 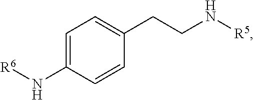
wherein R⁵ is selected from 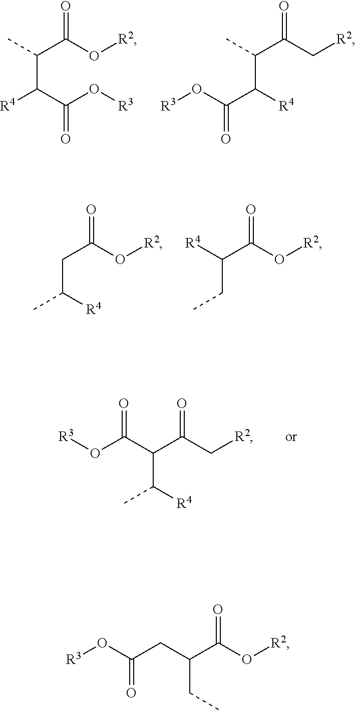
wherein R⁶ is selected from hydrogen, 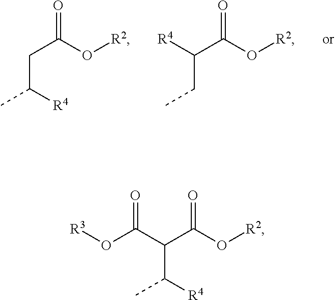
wherein R² and R³ are for each occasion independently selected from hydrogen, an alkyl, or an alkenyl, R⁴ is for each occasion independently selected from hydrogen, an alkyl, an alkenyl, an alkoxy, or an alkenoxy.

14. The two-component adhesive according to claim 13, wherein the component B is an isocyanate resin.

15. The two-component adhesive according to claim 14, wherein the isocyanate resin comprises hexamethylene diisocyanate, isophorone diisocyanate, toluene diisocyanate, methylene diphenyl diisocyanate, a isocyanurate trimers, a biurete, a uretdione dimers, or any combination thereof.
